(19) United States
(12) Patent Application Publication
(10) Pub. No.: US 2007/0290865 A1
(43) Pub. Date: Dec. 20, 2007

(54) Apparatus and method for assuring clean hands

(76) Inventors: John Lynn, Austin, TX (US); Gregory Terry Moore, Knoxville, TN (US)

Correspondence Address: HOUSTON OFFICE OF;NOVAK, DRUCE & QUIGG, LLP, 1000 LOUISIANA STREET, FIFTY-THIRD FLOOR, HOUSTON, TX, 77002, US

(21) Appl. No.: 11/805,235

(22) Filed: May 22, 2007

Related U.S. Application Data
(60) Provisional application No. 60/815,085, filed on Jun. 20, 2006.

(51) Int. Cl. G08B 23/00

(52) U.S. Cl. 340/573.1

(57) ABSTRACT

A method and apparatus for assuring clean hands are disclosed. The method includes marking a hand of a user with a portable marking mechanism which contains an easily identifiable substance that can be washed off a person's hands only with effective hand washing.

### CROSS REFERENCE TO RELATED APPLICATIONS

This application claims priority from provisional patent application 60/815,085 with a filing date of Jun. 20, 2006 and entitled “Apparatus and Method for Assuring Clean Hands”.

### TECHNICAL FIELD

The present disclosure relates in general to the field of hand hygiene, and, more particularly, to doing so by marking a person's hands using a portable marking device which marks with an easily identifiable substance that can only be washed off with efficacious hand washing.

### BACKGROUND

In a variety of different fields, including businesses and residences, there is a great desire to encourage people to wash their hands more effectively and more frequently. The U.S. Center for Disease Control has stated that the most important thing people can do to keep from getting sick is to wash their hands. And yet, many people, through lack of knowledge, poor habits or simple negligence either do not wash their hands frequently enough or effectively enough.

An example of a specific need for assuring sanitized hands is the food services industry. It has been known for many decades that food preparers, servers and so forth should clean and sanitize their hands prior to handling food to be served. This need is self-evident when food service employees enter restrooms. Bacteria (such as E-coli, those found in fecal matter, uncooked foods, etc.) in restrooms are well known health hazards and without proper cleaning/sanitization of the hands of restaurant employees, can be transmitted to unknowing customers. These and other concerns also suggest a need for sanitizing hands in private residences, especially in homes with children.

Currently, parents as well as health industry participants are trying to address the problem of insufficient hand washing with rules and regulations concerning hand washing. For instance, in many food service establishments there are signs which state roughly “Employees must wash their hands before leaving.” Methods that require adherence to a rule or policy by human beings with little or no reinforcement or monitoring, however, are typically insufficient to maximize compliance. Importantly, such rules and regulations do little or nothing to assure that even if hand washing is done, it is done correctly (e.g., with enough cleaning composition, for a long enough period of time to kill the germs, or with sufficient scrubbing).

Presently there are several systems intended to address the problem of insufficient hand washing. These systems are typically very complex and, accordingly, prohibitively expensive. U.S. Pat. No. 5,670,945, for example, discloses a complex system that has a sanitizing basin with moisture proof switches inside the sanitizing basin and proximity detectors. A person must insert both hands simultaneously into the sanitizing basin in order to initiate the desired output signal.

The GloGerm product works by marking the person's hand with a substance that glows when exposed to UV light sources. This product requires the purchase of the UV lamps that illuminate the marking substance and therefore it is not an easy to use anywhere. Additionally, U.S. Pat. Nos. 6,426,701; 5,945,910; 5,812,059; 5,202,666; 4,896,144; 3,967,478; 5,610,589; 4,688,585 and 5,199,188 and U.S. Patent Application Nos. 2003/0030562; 2003/0197122; 2003/0019536; and 2004/0001009 all involve relatively complex systems containing components such as complex electronics, location sensors, pumps and so forth (often mixed together in complex attempts to require hand washing). In summary, the presently available systems are typically not portable (i.e. used anywhere), expensive, complex to install, difficult to maintain and it can be difficult to train users in their operation

### SUMMARY

In accordance with the present disclosure, the disadvantages and problems associated with assuring clean hands have been substantially reduced or eliminated. In a particular embodiment, a hand of a user is marked with an easily identifiable substance with an easily portable marking device such as a handheld marker immediately before hand washing. The easily identifiable substance is designed such that it requires a good washing of the hands to remove it. Failure to wash one's hands well will be obvious since the easily identifiable substance is still observable.

### BRIEF DESCRIPTION OF THE DRAWINGS

A more complete and thorough understanding of the present disclosure and advantages thereof may be acquired by referring to the following description taken in conjunction with the accompanying drawings, in which like reference numbers indicate like features, and wherein:

FIG. 1 illustrates a cross-sectional view of one embodiment of a marker to mark one's hands according to teachings of the present disclosure;

FIG. 2 illustrates one of many marking patterns on a person's hand which may help assure that the hands are thoroughly washed.

### DETAILED DESCRIPTION

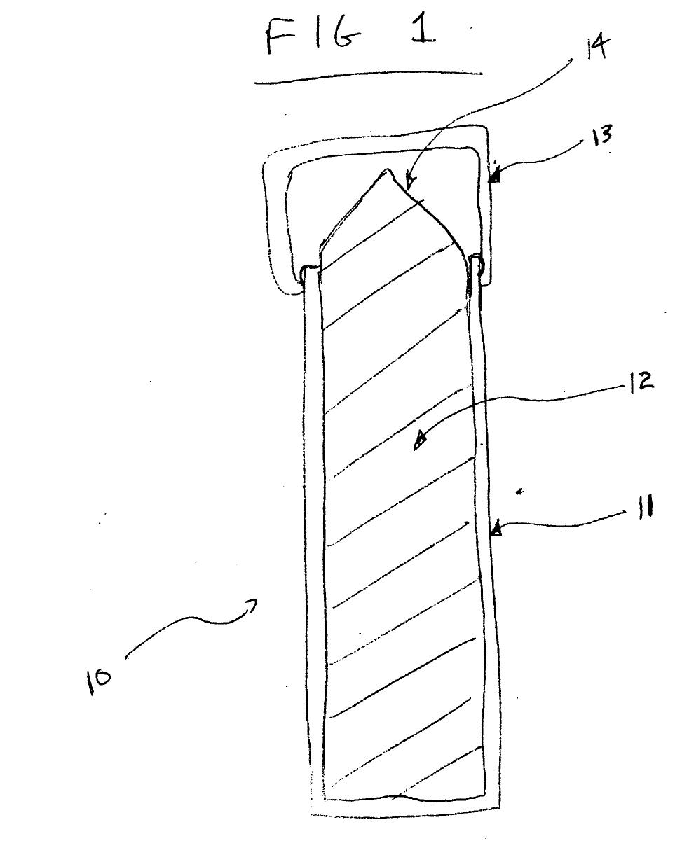

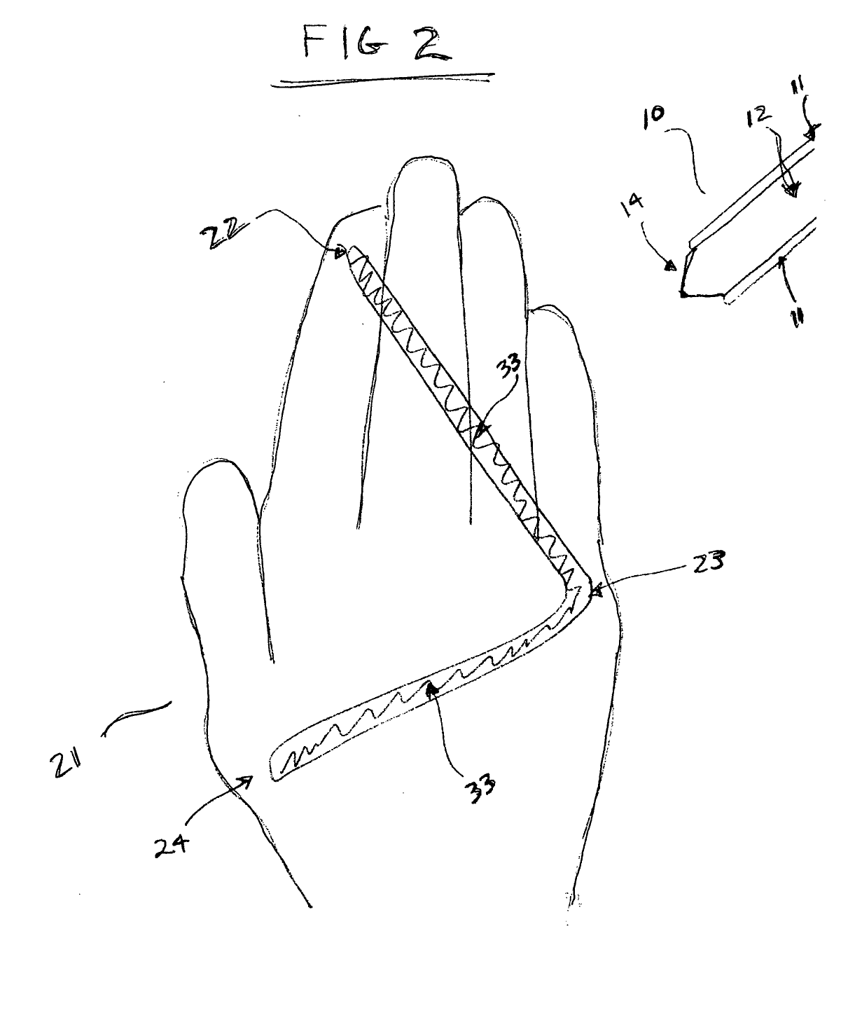

Preferred embodiments of the present disclosure and their advantages are best understood by references to FIGS. 1 and 2, where like numbers are used to indicate like and corresponding parts.

FIG. 1 is a cross-sectional view of one embodiment of a marker used to mark one's hand to assure that the hand's are washed well. In the embodiment of FIG. 1, marker 10 includes cylindrical container 11 which holds absorbent material 12. Absorbent material 12 is saturated with an easily identifiable substance (not shown since it is an integral part of the absorbent material 12). To help prevent the easily identifiable substance from drying out the marker may include a cap 13 which is fitted to be detachably attached to container 11.

The marker of this invention may be especially desirable for parents or teachers to use to assure that their children or students do a good job of washing their hands. It can be used very simply by the adult removing cap 13 which exposes protruding tip 14 of absorbent material 12. The parent or teacher then simply puts a mark (or marks) on the child's hand or hands.

The mark or marks would preferably be placed such that it helps to assure a thorough washing of the hands. FIG. 2 illustrates an example of a single long mark which helps accomplish a thorough washing of hand 21. Multiple marks in various locations on hand 21 may be desirable to assure a thorough hand washing but may take more time than a single mark with essentially one sweep of the marker. In FIG. 2 the mark from marker 10 is “V shaped” line 33 going from tip of index finger 22 to top of the palm at the base of the little finger 23 and then back to base of the palm underneath the thumb 24. To remove this mark the child would likely have to do a good job of washing their entire hand (and since it takes two hands to wash one hand the other unmarked hand will also be well washed).

There are many different configurations of a single long mark or a series of marks which will help assure clean hands and the example set forth in FIG. 2 is just one. The parent or teacher may also decide to mark both hands but this may not be necessary.

Absorbent material 12 may be any of a number of materials (e.g., felt used for markers or a pad used for stamps). One commercially available absorbent pad is felt “Fiber Engineered” manufactured by AFFCO of New Windsor, N.Y. Absorbent material 12 includes an easily identifiable substance used to mark a hand that comes in contact with absorbent material 12. Although a specific marking mechanism is illustrated in FIG. 1, the marking mechanism may also be implemented using a variety of technologies and configurations. The marking mechanisms may not be simple pen like marker but may include systems, including but not limited to: (1) mechanical, (2) pneumatic, (3) pneumatic (mechanical), (4) electronic, and (5) any combination thereof. Additionally, the marking mechanism may dispense the easily identifiable substance as a spray, micro-dot, gravity drop, squirt, print, stamp or mist.

The easily identifiable substance may be a variety of different types of substances such as those substances set forth in the U.S. Pat. Nos. 6,031,461 and 6,211,788. These substances must be non-toxic and may include inks, dyes, vegetable dyes, paint, stain, pigment, grease and any combination thereof. The easily identifiable substance may also be designed such that it is not easily identifiable visually, but instead is easily identifiable using other detection mechanisms such UV light, or using small metallic substances that are electronically detectable. The easily identifiable substance could further be additional substances such as extremely tiny integrated circuits (sometimes referred to as “smart dust”). Preferably the easily identifiable substance contains only FDA approved materials.

The easily identifiable substance may be selected or designed such that the desired type of hand washing is necessitated, once the substance is applied on a hand. For example, the easily identifiable substance may be designed such that it can only be removed by washing it off with a cleaning composition (e.g. soap) and hot or warm water for a sufficient period of time, thereby maximizing sanitation of a person's hands.

One example of an easily identifiable substance may be found in the ink in “Washable Markers CLASSIC” manufactured by Rose Art, Inc. This product comes in a variety of colors and experiments have shown that a preferred color is orange. In a cleansing experiment, the orange “CLASSIC” marker came off very slowly (over a minute) with water only, but came off in approximately fifteen (15) to twenty (20) seconds with soap and warm water.

Other easily identifiable substances include an aqueous ink which is Food, Drug and Cosmetic approved from Specialty Inks. This vegetable dye based acqueous ink can be found in the marker of SquidSoap a commercially available product from SquidSoap LP. To assure that the inks do not dry out if cap 13 is left off it may be desirable to add a hydrophilic substance like FDA approved glycerine.

Because of the typically short time between marking the hand and washing the hand, staining or smearing of clothes is not normally a large issue. However, it may be desirable for the mark to dry very quickly on the hand once it is marked to minimize smearing on other objects. The “Washable Marker's CLASSIC” by Rose Art is an example of a good ink choice because of how quickly the ink drys once put on the hand.

Another method of minimizing smearing of other objects is placement of the easily identifiable substance on an area of the hand which is unlikely to come into contact with other items. For example the mark may be placed in between the fingers on the web of the hand, on the palm of the hand or on the back of the hand.

In case there is accidental smearing of the easily identifiable substance on other objects, it may be desirable to formulate the easily identifiable substance such that it includes a composition which is easily removable from clothes and other items. An example of an easily identifiable substance that is described in U.S. Pat. No. 5,043,013 issued to Kluger et al. and entitled “Washable Ink Compositions.” U.S. Pat. No. 6,147,607 also sets forth various substances which are more easily washable than typical inks, dyes or pigments. The “Washable Marker's CLASSIC” by Rose Art is an example of one such commercially available product.

The notion of “dirtying” one's hands in order to subsequently get the hands more effectively clean may be counter-intuitive, but it results in especially clean hands if the easily identifiable substance, marker and placement of the mark or marks on the hand are well chosen.

Although the present disclosure has been described in detail, it should be understood that various changes, substitutions, and alterations can be made without departing from the spirit and scope of the disclosure as defined by the appended claims. The marker has been shown in a cylindrical shape such as a pen, pencil or magic marker but it could in fact be any shape that allows portability.

What is claimed is:

1. A method for helping to assure washing of a person's hands, comprising marking a hand of the person with a portable marking mechanism which contains an easily identifiable substance, said easily identifiable substance requiring an effective washing of the hands to remove it from the person's hand.

2. The method of claim 1 further comprising marking the person's hand in a pre-determined pattern designed to assure that all the various parts of the hand are washed.

3. The method of claim 1, wherein the marking of the person's hand is selected from the group consisting of drawing, spraying, micro-dotting, gravity dropping, printing, misting and squirting.

4. An easily portable marking mechanism for marking a person's hand which contains an easily identifiable substance, said easily identifiable substance designed to be removable from the person's hand only if the hand is effectively washed.

5. The easily portable marking mechanism of claim 4 wherein the easily identifiable substance comprises glycerin and a vegetable dye.
